(19) United States
(12) Patent Application Publication
(10) Pub. No.: US 2016/0120418 A1
(43) Pub. Date: May 5, 2016

(54) BLOOD PRESSURE MEASUREMENT DEVICE

(71) Applicant: MEDIETA OY, Helsinki (FI)

(72) Inventors: Niku Oksala, Tampere (FI); Sami Melkoniemi, Espoo (FI)

(21) Appl. No.: 14/895,892

(22) PCT Filed: Jun. 3, 2014
(86) PCT No.: PCT/FI2014/050445

§ 371 Date: Dec. 3, 2015

(30) Foreign Application Priority Data
Jun. 3, 2013 (FI) 20135614
Jul. 4, 2013 (FI) 20135736

(51) Int. Cl. A61B 5/021; A61B 5/00; A61B 5/0205

(52) U.S. Cl. CPC A61B 5/02141; A61B 5/0205; A61B 5/02125; A61B 5/6824; A61B 5/6831; A61B 2560/0257; A61B 2562/0247; A61B 2562/04; A61B 2562/0214; A61B 2562/0219; A61B 2560/0223; A61B 2562/028

(57) ABSTRACT

A method for measuring blood pressure of a patient continuously and non-invasively comprises measuring a first pressure signal being proportional to arterial blood pressure of the patient at a first location by a first pressure sensor (P1). In addition ambient pressure is measured by a third ambient pressure sensor (P3). Furthermore the method comprises subtracting the signal derived from the third ambient pressure sensor from the signal derived from the first pressure sensor to compensate for alterations induced by alterations in measurement point altitude and atmospheric pressure changes thereby providing a signal representing relative systemic arterial blood pressure of the patient

### TECHNICAL FIELD OF THE INVENTION

The invention relates to a method and device for blood pressure measurement. Especially the invention relates to continuous non-invasive blood pressure measurement system based on pulse wave velocity (PWV) measurement.

### BACKGROUND OF THE INVENTION

Blood pressure is conventionally measured by devices relying on a tourniquet technology resulting in intermittent measurement. The intermittent measurement has several disadvantages, namely it is slow and cumbersome and in addition it blocks the blood circulation for the measurement. Also some continuous measurement systems are known based on a determination of Pulse Wave Velocity (PWV) and Pulse Transmit Time (PPT) measurements, where the pulse propagating in the blood vessel is determined and based on the wave velocity the blood pressure can be determined. However, the results of these continuous measurement systems are not typically very reliable due to e.g. motion of a patient to be measured or due to changing environmental factors, such as changing measurement altitude or pressure.

### SUMMARY OF THE INVENTION

An object of the invention is to alleviate and eliminate the problems relating to the known prior art. Especially the object of the invention is to provide a device for measuring blood pressure continuously and non-invasively in a reliable, easy and fast way. In addition the object of the invention is to make possible to calibrate the measurement without external devices.

The object of the invention can be achieved by the features of independent claims.

The invention relates to a device for measuring blood pressure of a patient continuously and non-invasively according to claim 1, as well as to a method according to claim 13.

According to an embodiment of the invention a device for measuring blood pressure of a patient continuously and non-invasively comprises a first pressure sensor (P1) configured to measure a pressure signal being proportional to arterial blood pressure of the patient at a first location, and a third ambient pressure sensor (P3) configured to measure ambient pressure. In addition the device is configured to subtract the signal derived from the third ambient pressure sensor from the signal derived from the first pressure sensor to compensate for alterations induced by alterations in measurement point altitude and atmospheric pressure changes thereby providing a signal representing relative systemic arterial blood pressure of the patient.

According to an embodiment the device further comprises also a second pressure sensor (P2) configured to measure a pressure signal being proportional to arterial blood pressure of the patient at a second location, wherein the device is configured to subtract the signal derived from the third ambient pressure sensor from the signal derived from the second pressure sensor to compensate for alterations induced by alterations in measurement point altitude and atmospheric pressure changes.

Furthermore according to an example two pressure sensors, advantageously capacitive sensors, are arranged in a single specifically engineered component to be located on the course of distal radial artery at a known distance from each other (p1, p2). Capacitive sensors have greater dynamic range and are more sensitive to compared to resistive sensors. This distance is optimized and minimized (1-4 cm) so that measurements can be done in a very small area in between the distal antebrachium and carpal area, at a location insensitive to movement artifact and so that all the measurements can be performed rapidly monitoring the propagation of arterial pulse wave. Therefore, the sensors can be placed in a comfortable and durable wristband.

In addition when the device with said sensors is to be placed on the course of distal radial artery and advantageously in the relatively small area between the distal antebrachium and carpal area, the measurement is very reliable because both sensors (P1, P2) measures accurately the same wave pulse from the same arterial vessel with the same viscoelastic properties. By this feature the measurements can be done very accurately and reliable manner for example when comparing to embodiment where the first sensor is at the wrist area and the second one is at the finger, namely the properties of the vessels at these different areas are different and also the pulse wave is different in the finger area than in the wrist area. In addition when the measurement points locate at the different sides of joints, the distance between the sensors will easily vary. All these above mentioned points induce inaccuracies and disadvantages to the measurement.

In addition to these, the device, advantageously the wristband may be equipped with at least two accelerometers, such as 3D MEMS accelerometers, capable of detecting the movements of the upper limb. These sensors (including also accelerometers) are advantageously located within 1-4 cm from each other. In addition one axis of the accelerometer is advantageously oriented along the axis of the upper limb.

According to an embodiment the contact of the pressure sensors with skin is mediated by elastic liquid filled pads composed of horizontal lamellar structures which allow efficient transfer of arterial pressure wave to sensors but eliminates transmission of pressure peaks caused by movement artifacts from other directions.

The sampling resolution is set at a frequency up to 1 kHz allowing accurate detection of pulse wave velocity. Therefore, the system is sensitive to rapid beat to beat alterations in arterial pulse wave propagation. This architecture has several benefits compared to previous ones. Conventional non-invasive blood pressure measurement relies on tourniquet technology resulting in intermittent measurement. The conventional PWV solutions rely on electrocardiographic synchronization of the measurement by r-wave and detection of the pulse wave by a peripheral sensor. These solutions are cumbersome, require sensors in multiple locations and are prone to significant bias due to two important reasons. Firstly, there is significant variation in the initiation of the actual cardiac ejection phase, pulse wave and r-wave. Secondly, the conventional methodology also requires estimation of the distance from heart to the peripheral measurement point which cannot be done accurately due to large anatomical variations.

The present invention offers also other advantages over the known prior art, such as the possibility to perform continuous and non-invasive blood pressure measurements. This is based on pulse wave velocity (PWV) measurement with continuous automatic calibration.

In addition the invention makes it possible to measure continuous non-invasive blood pressure by two exemplary ways and also makes it possible to calibrate the measurement without external devices, wirings or electrocardiographic electrodes at the initiation of the measurement and also to compensate for changes due to postural changes or movement. It also has the capability of continuous measurement in case of failure of one of the arterial sensors.

According to an embodiment the invention makes it possible to measure blood pressure via two closely placed sensors detecting pulse wave velocity (PWV) and third sensor compensating for fluctuations in pulse wave amplitude or speed due to postural changes or movement. In addition according to second embodiment the invention makes it possible to measure blood pressure directly via difference in arterial pressure sensors (only one needed) and separate ambient pressure sensor.

### BRIEF DESCRIPTION OF THE DRAWINGS

Next the invention will be described in greater detail with reference to exemplary embodiments in accordance with the accompanying drawings, in which:

FIGS. 1A-1C illustrate a principle of an exemplary device for measuring blood pressure of a patient continuously and non-invasively according to an advantageous embodiment of the invention,

FIG. 2 illustrates an exemplary method for measuring blood pressure of a patient continuously and non-invasively according to an advantageous embodiment of the invention, and

FIG. 3 illustrates another exemplary method for measuring blood pressure of a patient continuously and non-invasively according to an advantageous embodiment of the invention.

### DETAILED DESCRIPTION

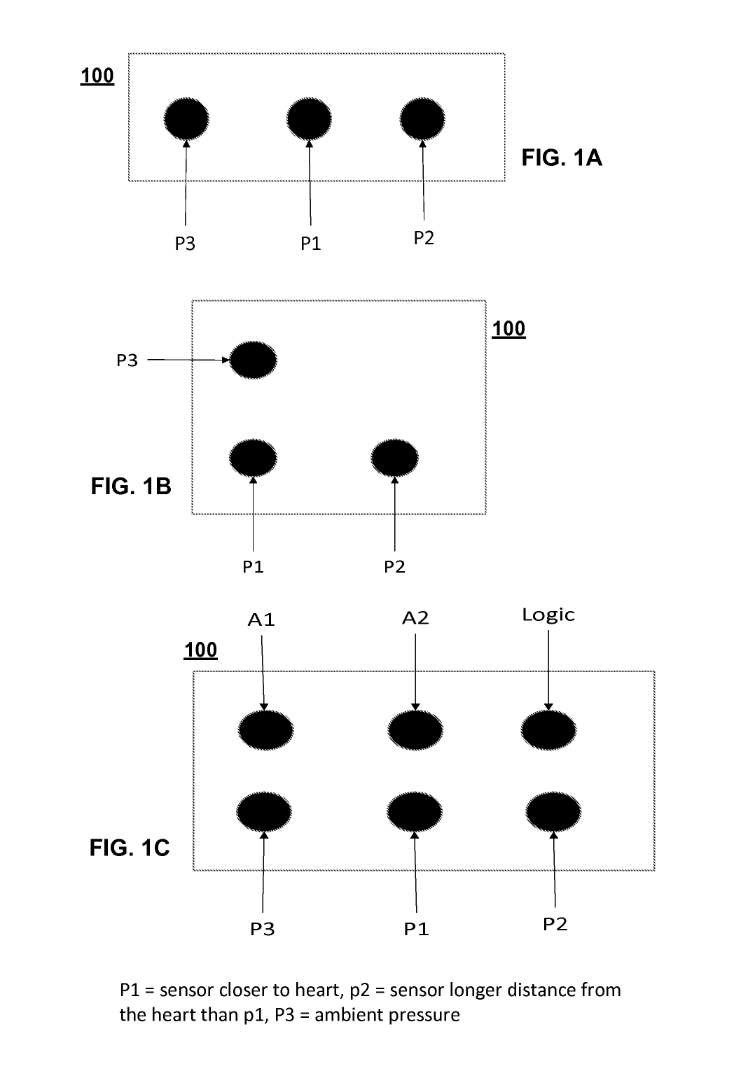

FIGS. 1A-1C illustrate a principle of an exemplary device 100 for measuring blood pressure of a patient continuously and non-invasively according to an advantageous embodiment of the invention, where the device comprises a first pressure sensor (P1) configured to measure a pressure signal being proportional to arterial blood pressure of the patient at a first location. The device comprises also a third ambient pressure sensor (P3) configured to measure ambient pressure. In addition the device is configured to subtract the signal derived from the third ambient pressure sensor from the signal derived from the first pressure sensor to compensate for alterations induced by alterations in measurement point altitude and atmospheric pressure changes thereby providing a signal representing relative systemic arterial blood pressure of the patient. It is to be understood that the subtraction and also other signal processing may be made in backend system, whereupon the device comprises advantageously wireless data communication means for communicating measurement signal to the backend.

The device may comprise also a second pressure sensor (P2) configured to measure a pressure signal being proportional to arterial blood pressure of the patient at a second location, whereupon the device is configured to subtract the signal derived from the third ambient pressure sensor from the signal derived from the second pressure sensor to compensate for alterations induced by alterations in measurement point altitude and atmospheric pressure changes.

Advantageously the first and second sensors are arranged to detect the signals so that the first proximal sensor detects the signal before the second distal one, whereupon the device is configured to provide this as a first quality control.

In addition the device 100 may comprise at least one, preferably two accelerometers A1, A2 for detecting movements of the user, such as movements of the hand or other changes in altitude, i.e. falls and collapses, as is described in FIG. 1C. The device may be configured to detect these movements based on the changes in detected pressure signals possibly supplemented by the measurements of said accelerometers, or alternatively based signals purely detected by said accelerometers. The accelerometers are advantageously 3D MEMS accelerometers. It is to be noted that the device, such as the wristband, additionally comprises also other components allowing the measurements, such as an MCU or ASIC logic circuit (logic), power source, like a battery, or the like.

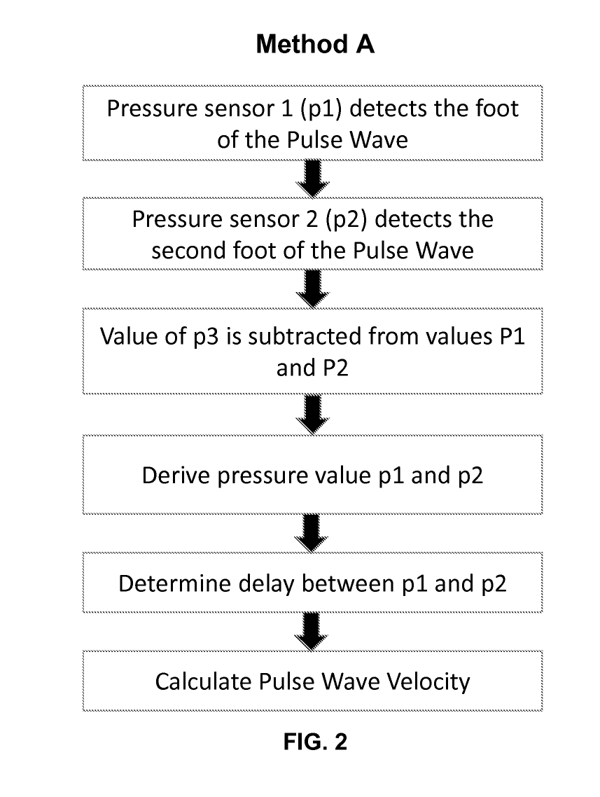

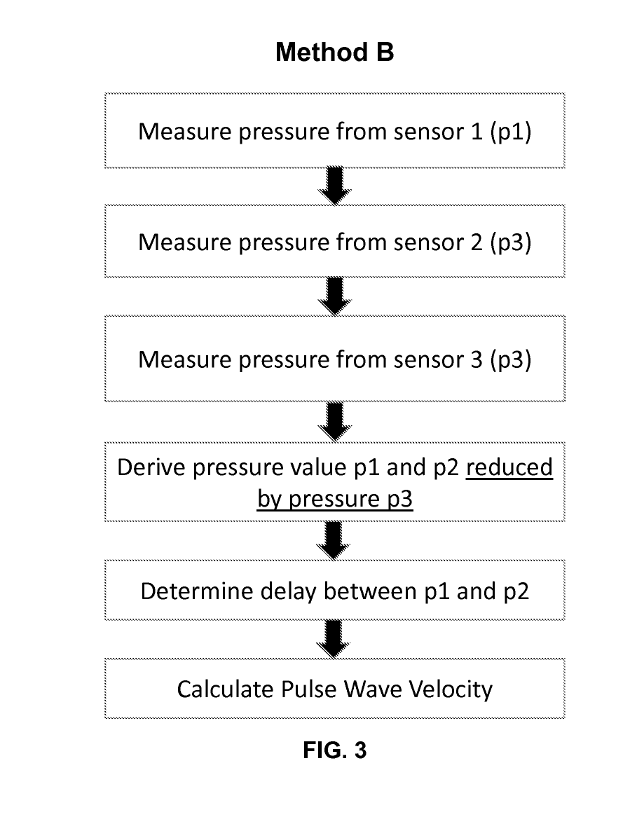

FIGS. 2 and 3 illustrate exemplary methods (Method A, Method B) for measuring blood pressure of a patient continuously and non-invasively according to advantageous embodiments of the invention.

Method A as described in FIG. 2.

Utilizing signal processing system, the sensors P1, P2 are placed so, that a maximum signal is derived and that both arterial pressure sensors P1, P2 detect the signals so that the proximal sensor fires before the distal one. This procedure provides the first quality control. A third capacitive pressure sensor P3 advantageously with equal characteristics is utilized to measure the ambient pressure signal. The signal derived from the ambient pressure sensor P3 is subtracted from signals derived from the arterial sensors P1, P2 to compensate for alterations induced by alterations in measurement point altitude (i.e. postural changes, alterations in measurement point position relative to heart) and atmospheric pressure changes. This signal can yield changes in altitude with a resolution of centimetres and therefore measure the changes in the vertical position of the arterial pressure sensors. For example, if the ambient pressure suddenly rises or decreases (i.e during movement of upper limb, climbing of stairs or opening or closing of doors), this is immediately reflected also in the arterial sensor readings and amplitude of the pulse wave.

Utilizing the embodiments of the invention the signal to noise ratio can be maximized continuously. For example, raising the hand above the head results in greatly lowered amplitude of the pulse wave in addition to obvious slowing down of the PWV. This makes it hard to reliably detect the critical phases of the wave (i.e. the foot-phase of the pulse wave) needed for accurate PWV calculation. One of the primary interests of the invention is to derive the systemic arterial pressure of which the pressure reading at the wrist is an approximation. The third sensor P3 reading can also be used to extrapolate the systemic pressure since in addition to the initial calibration procedure (see below, yielding the distance from heart level to wrist area) it makes it possible to continuously detect the changes in measurement point height during patient movement and compensate the readings accordingly. It can also be utilized to model rapid changes in altitude, i.e. falls and collapses.

In addition, according to an embodiment movements of the hand or other changes in altitude, i.e. falls and collapses, can be additionally or independently detected by accelerometers (such as 3D MEMS accelerometers), which can be configured to be capable of detecting upper limb movements and providing signals indicating walking, standing, sitting and laying supine, as an example.

Method B as described in FIG. 3.

Theoretically, after subtraction of the ambient pressure value, the arterial pressure sensors can provide arterial pressure values, at least when calibrated with subsequent calibration procedure (see below) and individual fitting of the algorithm. However, this is largely dependent on the pressure by which the sensors are compressed against the artery (i.e. mounting pressure). Since this is hard to standardize, these readings are not considered reliable as absolute values but can be utilized for the detection of significant relative changes in arterial pressure given that the mounting pressure is held constant. This method utilizes either P1 or P2 and the sensor P3. It can yield values in case only one of the sensors (P1/P2) is functioning correctly and provides a reserve measurement method which can detect significant relative changes in blood pressure.

Baseline Calibration Procedure

The ambient pressure sensor is used for baseline calibration. Blood pressure measurement should be performed so that the measurement point stays at a constant distance from heart. The ambient pressure sensor can yield the change in vertical displacement or altitude relative to sea level at a resolution of few centimeters as atmospheric pressure is a function of altitude. Therefore, the system automatically calibrates to different measurement conditions, regardless of altitude. This provides a second quality control (C2). To convert relative measures to absolute ones, a patient specific calibration procedure is performed so that when lying supine, the upper limb is raised straight at an angle of 90° relative to the horizontal plane. This procedure can be monitored, according to an exemplary embodiment, by the accelerometers (e.g. 3D MEMS accelerometers) and the PWV calculation algorithm is executed when the 90° angle is achieved. Using the equation (1), where Δh is the altitude change, ρ is the density of blood which is considered constant and g is the gravitational constant the absolute change in hydrostatic pressure (ΔP_{hydrostatic}) calculated:

ΔP_{hydrostatic}=Δhρg (1)

Using this equation, the pressure values from arterial sensors can be calibrated to absolute values. This provides a third quality control (C3). This procedure also yields the approximate distance Δh from body to wrist to be utilized in continuous auto calibration sequences. The changes in ambient temperature in this context are considered not significant. To yield another, potentially more reliable measure of arterial pressure, two other parameters are derived. The time needed (i.e. pulse transit time PTT) for the pulse wave to propagate from proximal sensor to distal sensor (p1, p2) is calculated by a mathematical algorithm tracking a specific point at the foot of the pulse wave known to be insensitive to reflections of the pulse wave. The result is the pulse wave velocity (PWV) and PTT. Alterations in PWV and PTT have been shown to correlate well with alterations in systemic arterial pressure. However, interpersonal correlation is weaker. The signal processing algorithm may be integrated in the signal processing unit of the component itself or located in a remote backend system.

The absolute pressure values are derived by first utilizing the Moens-Korteweg equation (2), where t is the thickness of the artery wall, d is the diameter of the artery, ρ is the density of blood which is considered constant, and E is the Young's modulus reflecting the elasticity of the arterial wall. This equation can also be used to derive E, a parameter which associates with probability of future cardiovascular events when PWV is known:

$$P W V = \sqrt{\frac{tE}{ρ d}} \qquad ( 2 )$$

The Young's modulus E is not constant but varies with pressure. The dependence of E on pressure is shown by equation (3), where E₀ is the zero pressure modulus, α is a vessel constant (experimentally validated α=0.017 mmHg⁻¹), P is pressure and e is the Euler number (2.71828 . . . ):

E=E₀e^{αP} (3)

When equation (2) is substituted to (3) it yields equation (4) which describes the association of PWV with P and zero pressure elasticity E₀.

$$P W V = \sqrt{\frac{{tE}_{0} {e}^{α P}}{ρ d}} \qquad ( 4 )$$

From this equation, P can be solved:

$${PWV}^{2} = \frac{{tE}_{0} {e}^{α P}}{ρ d} \qquad ( 5 )$$

Of specific importance is that from this equation E₀ or subsequently E can also be solved then describing the association of zero pressure elasticity or Young's modulus E and PWV when pressure P is known, derived either by external measurement device or previously described method (A) which can be utilized with adequate accuracy at least when the measurement is performed under constant mounting pressure conditions (E₀=PWV²ρd/[te^{αP}] or E=PWV²ρd/t). These parameters can be utilized in the prediction of future cardiovascular events or in the monitoring of treatment response.

$$\begin{matrix}\frac{ρ {dPWV}^{2}}{t {E}_{0}} = {e}^{α P} & ( 6 ) \\ ln ( \frac{ρ d}{t {E}_{0}} {PWV}^{2} ) = ln {e}^{α P} & ( 7 ) \\ ln ( \frac{ρ d}{t {E}_{0}} {PWV}^{2} ) = α P & ( 8 ) \\ ln ( \frac{ρ d}{t {E}_{0}} ) + ln ( {PWV}^{2} ) = α P & ( 9 ) \\ ln ( \frac{ρ d}{t {E}_{0}} ) + 2 ln ( P W V ) = α P & ( 10 )\end{matrix}$$

Of specific importance is that from equation (10) α can be easily solved when P and PWV are known.

$$\begin{matrix}P = \frac{1}{α} ln ( \frac{ρ d}{t {E}_{0}} ) + \frac{2}{α} ln ( P W V ) & ( 11 ) \\ P = K + \frac{2}{α} ln ( P W V ) \\ with K = \frac{1}{α} ln ( \frac{ρ c}{t {E}_{0}} ) & \begin{matrix}( 12 ) \\ ( 12 )\end{matrix}\end{matrix}$$

From the equation (12) one can see that pressure is easily derived taken that the constant K is obtained. During the calibration procedure, equation (1) holds and the absolute value of ΔP_{hydrostatic} is known since Δh is directly obtained from the ambient pressure sensor (or from the accelerometer data, as is disclosed elsewhere in this document):

ΔP_{hydrostatic}=Δhρg (1)

During calibration procedure, the hydrostatic pressure changes when the upper limb is raised. Substituting equation (1) into equation (12) yields:

$$\begin{matrix}Δ {P}_{hydrostatic\_calibration} = K + \frac{2}{α} ln ( Δ {PWV}_{calibration} ) & ( 13 ) \\ K = Δ {P}_{hydrostatic\_calibration} - \frac{2}{α} ln ( Δ {PWV}_{calibration} ) & ( 14 )\end{matrix}$$

Therefore, the patient-specific and measurement-specific constant K can be obtained during the calibration procedure. The optimal procedure is to first determine K during calibration procedure using equation (14), then substituting K into equation (12) giving the pressure P as a function of PWV.

$$P = Δ {P}_{hydrostatic\_calibration} - \frac{2}{α} ln ( Δ {PWV}_{calibration} ) + \frac{2}{α} ln ( P W V ) \qquad ( 15 )$$

Changes in the position of the upper limb relative to body cause alterations in hydrostatic pressure. These changes can be compensated easily since the ambient pressure sensor P3 continuously reports the changes in height. These considerations apply only when the system is used at constant altitude since there is no body reference altitude sensor. Therefore, the system may be built so that the equation (15) is substituted with a hydrostatic pressure term (ΔP_{hydrostatic}_{_}_{calibration}) correcting for upper limb position alterations relative to heart. This term is either positive or negative depending on the altitude change relative to default set point determined during baseline calibration:

$$P = Δ {P}_{hydrostatic\_calibration} - \frac{2}{α} ln ( Δ {PWV}_{calibration} ) + \frac{2}{α} ln ( P W V ) + Δ {P}_{hydrostatic\_position} \qquad ( 16 )$$

Alternatively or as a supplementary function, the baseline calibration procedure yielding Δh and ΔP_{hydrostatic}_{_}_{calibration} and subsequently ΔPWV_{calibration} can be done utilizing the two accelerometers. According to an embodiment this can be implemented even without the pressure sensor P3. As one of the three 3D accelerometer axes in both accelerometers is positioned perpendicular to the wristband and parallel to axis of the upper limb, it is therefore capable of measuring the centrifugal or radial accelerations a₁ and a₂ at distances r₁ (the proximal accelerometer) and r₂ (the distal) along the axis of the upper limb.

In the following equation, the radial accelerations at the specified two measurement locations where ω is the angular velocity are:

a₁=ω²r₁ and a₂=ω²r₂ (17)

The difference in acceleration between the two accelerometers is:

a₂−a₁=ω²−ωr₂−ω²r₁ (18)

Subsequently, let D be the fixed distance between the two accelerometers (D=r₂−r₁):

a₂−a₁=ω²(r₂−r₁) (19)

which yields the angular velocity of the upper limb:

ω=[(|a₂−a₁|)/D]^{1/2} (20)

The radius r=(r₂+r₁)/2 at the center of the wristband which equals Δh when the upper limb is flexed at 90° angle relative to the vertical axis of the patient when standing erect or sitting, i.e. strictly horizontally, can then be calculated. The centrifugal force at the center of the wristband during rigorous horizontal swing of the upper limb can be calculated:

F=(mω²)/r (21)

r=(mω²)/F, (22),

where F=ma, and m is the mass of the accelerometer sensor element which is the same in both accelerometers and therefore their average is simply m, where a is the acceleration (a₂+a₁)/2 at the center of the wristband

r=ω²/a (23)

Substituting equation (20) into (23) yields:

r=[(|a₂−a₁|)/D]/a, (24)

r=[(|a₂−a₁|)/D]*2/(a₂+a₁) (25),

and r=Δh

r=2(|a₂−a₁|)/[D(a₂+a₁)] (26)

Subsequently, when the upper limb is flexed at 90° position relative to the plane when the patient is lying supine, the ΔPWV_{calibration} is recorded simultaneously with ΔP_{hydrostatic}_{_}_{calibration} and the values processed as described before.

Utilizing the pulse wave curve, an algorithm can be utilized to derive heart rate as number of pulse waves per time unit, respiratory rate from baseline, amplitude and heart rate variability using wavelet transform function.

### Continuous Auto Calibration Procedure

In method A the subtraction of P3 reading from P1 and P2 results in stable amplitude and maximal signal-to-noise ratio. The readings from P3 can be used to detect changes measurement point altitude and therefore movement of wrist relative to heart level during movement or postural changes. This data can also be used to extrapolate systemic pressure levels as described earlier since the Δh is obtained during baseline calibration sequence.

In method B the subtraction of P3 reading results in absolute arterial pressure values. The readings from P3 can be used to extrapolate systemic pressure levels or compensate for movement or postural changes.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims. For example it is to be noted that, analogously as in the baseline calibration procedure, the accelerometer sensor output yielding the angular velocity w and tilt of the upper limb can be used for continuous autocalibration. In addition it is to be noted that the accelerometers described above may be e.g. 3D MEMS accelerometer or similar known from the prior art.

In addition it is to be noted that the device can be advantageously implemented by a wristband device, where the wristband device comprises advantageously all the pressure and acceleration sensors. The data processing can also be implemented by the wristband device, or alternatively the wristband device may send (e.g. wireless way) the measuring signals to the external data processing backend for data calculation. The data processing backend may comprise e.g. could server, any computer or mobile phone application and according to an example it can send the calculated results or otherwise processed data e.g. for displaying back to the wristband device or other data displaying device, such as a computer or the like in data communication network or to a smartphone of the user.

1. A device for measuring blood pressure of a patient continuously and non-invasively, wherein the device comprises:
a first pressure sensor configured to measure a pressure signal being induced by arterial blood pressure of the patient at a first location,
a second pressure sensor configured to measure a pressure signal being induced by arterial blood pressure of the patient at a second location, said second location being different than said first location,
a third ambient pressure sensor configured to measure ambient pressure,
wherein the device is configured to:
subtract the signal derived from the third ambient pressure sensor from the signal derived from the first pressure sensor and the from the signal derived from the second pressure sensor to compensate for alterations induced by alterations in measurement points altitude and atmospheric pressure changes thereby providing a signal representing relative systemic arterial blood pressure of the patient.

2. The device of claim 1, wherein the first and second sensors are arranged in the device so that in use they are configured to be pressed against measurement location of the patient at a known fixed distance from each other, wherein the distance is between 0.5-5 cm.

3. The device of claim 2, wherein the device is configured to be placed on a measurement location on the course of distal radial artery.

4. The device of claim 1, wherein the pressure sensors are capacitive sensors.

5. The device of claim 1, wherein the device comprises at least one accelerometer for measuring movements of the device and thereby the movements of the user, comprising movements of a hand or changes in altitude, comprising falls and collapses.

6. The device of claim 14, wherein the device is configured to use acceleration data provided by at least two accelerometers for baseline calibration procedure yielding Δh and ΔP_{hydrostatic}_{_}_{calibration} and subsequently ΔPWV_{calibration}.

7. The device of claim 1, wherein the pressure sensors are capacitive pressure sensors, and wherein the sampling resolution is at least 100 Hz.

8. The device of claim 1, wherein a maximum signal is derived of the pulse wave after said subtraction and wherein the first and second sensors are arranged to detect the signals so that the first proximal sensor detects the signal before the second distal one, whereupon the device is configured to provide this as a first quality control.

9. The device of claim 1, wherein the blood pressure is based on pulse wave velocity measurement, wherein the velocity of the pulse is determined based on the time difference between said first and second detectors detect the same pulse and the distance of said first and second sensors.

10. The device of claim 1, wherein the signal of the third ambient pressure sensor is used for calibration of the first or second sensors measurements so that the signals representing the absolute systemic arterial blood pressure of the patient is provided.

11. The device of claim 1, wherein the resolution of the third ambient sensor is proportional to a change in vertical displacement or altitude relative to sea level at a resolution of few centimetres.

12. A wristband device for measuring blood pressure of a patient continuously and non-invasively, wherein the wristband device comprises:
a first pressure sensor configured to measure a pressure signal being induced by arterial blood pressure of the patient at a first location,
a second pressure sensor configured to measure a pressure signal being induced by arterial blood pressure of the patient at a second location, said second location being different than said first location,
a third ambient pressure sensor configured to measure ambient pressure,
wherein the wristband device is configured to:
subtract the signal derived from the third ambient pressure sensor from the signal derived from the first pressure sensor and the from the signal derived from the second pressure sensor to compensate for alterations induced by alterations in measurement points altitude and atmospheric pressure changes thereby providing a signal representing relative systemic arterial blood pressure of the patient,
or
send said measured signals to a backend data processing unit for subtracting the signal derived from the third ambient pressure sensor from the signal derived from the first pressure sensor and the from the signal derived from the second pressure sensor to compensate for alterations induced by alterations in measurement points altitude and atmospheric pressure changes thereby providing a signal representing relative systemic arterial blood pressure of the patient.

13. A method for measuring blood pressure of a patient continuously and non-invasively, wherein
a first pressure signal being proportional to arterial blood pressure of the patient at a first location is measured by a first pressure sensor,
a second pressure signal being proportional to arterial blood pressure of the patient at a second location is measured by a second pressure sensor, where said second location is different than said first location,
ambient pressure is measured by a third ambient pressure sensor,
wherein the method further comprises:
subtracting the signal derived from the third ambient pressure sensor from the signal derived from the first pressure sensor and from the signal derived from the second pressure sensor to compensate for alterations induced by alterations in measurement points altitude and atmospheric pressure changes thereby providing a signal representing relative systemic arterial blood pressure of the patient.

14. The device of claim 5, wherein said accelerometer is a 3D MEMS accelerometer.

15. The device of claim 11, wherein the resolution is 2 or less centimeters.
